(19) United States
(12) Patent Application Publication
(10) Pub. No.: US 2007/0249011 A1
(43) Pub. Date: Oct. 25, 2007

(54) Method for Screening Fungus-Specific Antimicrobial Agent and Kit Therefor

(75) Inventors: Toshiaki Kudo, Saitama (JP); Takayuki Motoyama, Saitama (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: RIKEN, Wako-shi, Saitama (JP)

(21) Appl. No.: 10/591,464

(22) PCT Filed: Mar. 4, 2005
(86) PCT No.: PCT/JP05/04272

§ 371 Date: Sep. 1, 2006

(30) Foreign Application Priority Data
Mar. 4, 2004 (JP) 2004-061273

(51) Int. Cl. C12N 1/16; C12Q 1/18

(52) U.S. Cl. 435/32; 435/254.2

(57) ABSTRACT

An object of the present invention is to provide a method for effectively screening a agricultural chemical candidate or drug candidate. Specifically, an object of the present invention as to a agricultural chemical is to effectively screen, without the use of plants, a agricultural chemical candidate compound that does not exert a harmful effect on other organisms such as plants. The present inventors have completed the present invention by successfully developing a screening method comprising transforming, with an expression vector of a filamentous fungus-specific enzyme-encoding gene, a yeast that is biologically closely related to a filamentous fungus but does not have the enzyme, and applying agricultural chemical candidate samples to a control yeast (which does not express a filamentous fungus-specific enzyme) and to the filamentous fungus-specific enzyme-expressing transformant to select, as an agricultural chemical candidate, a agricultural chemical candidate sample that does not exert an effect such as a side effect on the control yeast and exhibits growth inhibitory or fungicidal activity specific only for the filamentous fungus-specific enzyme-expressing transformant. This screening method can also be used in the screening of a drug candidate.

### TECHNICAL FIELD

The present invention relates to a novel method for screening a filamentous fungus-specific antimicrobial agent, for example filamentous fungus-specific agricultural chemical or anti-filamentous fungus drug. More specifically, the present invention relates to a method for screening a reagent that is targeted for a filamentous fungus-specific enzyme.

### BACKGROUND ART

Conventional tests conducted for the purpose of searching substances useful as agricultural chemicals typically perform a primary test on media in laboratories. Such tests for target pathogenic microbes on media catch all of various reagents effective for the target pathogenic microbes. Therefore, there is high possibility of picking up a reagent that exhibits microbicidal properties even for nontarget organisms.

On the other hand, pathogen control tests using a portion of crops or plant pots in greenhouses indispensably involve the raising and preparation of crops and require much expense and work. Therefore, the tests are unsuitable for the examination of a very large number of test compounds because the number of compounds serving as novel functional regents are now said to be about 1/10000 that of test compounds (Non-Patent Document 1).

Furthermore, in such methods, side-effects and the like on plants and humans could not sufficiently be studied.

For example, organic mercury agents had been used as important rice blast control agents. However, the use of the agents was discontinued in consideration of toxicity to animals.

Moreover, a method using a cultured plant cell has been proposed as a method for conveniently examining the plant toxicity of agricultural chemicals (Patent Document 1: JP Patent Publication (Kokai) No. 5-294995A (1993)). Alternatively, Patent Document 2 (JP Patent Publication (Kokai) No. 9-124411A (1997)) has described a method for screening phytoalexin for a blast control agent comprising adding dropwise a test sample to the tip of a rice leaf and after 1 week, cutting the leaf, followed by extraction to confirm the presence or absence of generation of phytoalexin by HPLC.

On the other hand, the analysis of the sites of action of reagents phenylpyrroles, dicarboximides, and aromatic hydrocarbons known as agricultural chemicals effective for a variety of filamentous fungal diseases (Non-Patent Documents 2 to 7: Pillonel and Meyer, 1997; Zhang et al., 1999; Fujimura et al., 2000; Ochiai et al., 2001; Ochiai et al., 2002; and Oshima et al., 2002) has revealed that targets of many filamentous fungus-specific agricultural chemicals are filamentous fungus-specific histidine kinase (hereinafter, referred to as Os-1 family histidine kinase). In the present specification, the Os-1 family histidine kinase (also expressed as an Os-1 family, which was also expressed as an Os-1 subfamily) means hybrid-type histidine kinase having a histidine kinase domain and response regulator domain derived from fungi (filamentous fungi) performing mycelial growth, and having a region exhibiting 50% or more homology to the full length of a region containing six amino acid repeats (amino acid repeat 1 (171-260, 90 aa), amino acid repeat 2 (261-352, 92 aa), amino acid repeat 3 (353-444, 92 aa), amino acid repeat 4 (445-536, 92 aa), amino acid repeat 5 (537-628, 92 aa), and amino acid repeat 6 (629-700, 72 aa)) that exist in Neurospora crassa hybrid-type histidine kinase Os-1 (SEQ ID NO: 17; also referred to as Nik-1; Non-Patent Document 8: Alex et al., 1996 Proc. Natl. Acad. Sci. USA 93: 3416-3421) and exhibit homology to each other.

The screening of a drug also presents similar problems.

Patent Document 1: JP Patent Publication (Kokai) No. 5-294995A (1993)

Patent Document 2: JP Patent Publication (Kokai) No. 9-124411A (1997)

Non-Patent Document 1: Phytopathological encyclopedia, Yokendo, Mar. 30, 1995, p. 783-784

Non-Patent Document 2: Pillonel, C., and Meyer, T. 1997. Effect of phenylpyrroles on glycerol accumulation and protein kinase activity of Neurospora crassa. Pestic. Sci. 49: 229-236

Non-Patent Document 3: Ochiai, N., Fujimura, M., Oshima, M., Motoyama, T., Ichiishi, A., Yamada-Okabe, H. and Yamaguchi, 1. 2002. Effects of iprodione and fludioxonil on glycerol synthesis and hyphal development in Candida albicans. Biosci. Biotechnol. Biochem. 66: 2209-2215.

Non-Patent Document 4: Zhang, Y., Lamm, R., Pillonel, C., Xu, J.-R., and Lam, S. 1999. The hyper-osmotic stress response pathway of Neurospora crassa is the target of phenylpyrrole fungicides. Proc. 20th Fungal Genetics Conference, Asilomar, USA, p. 72

Non-Patent Document 5: Fujimura, M., Ochiai, N., Ichiishi, A., Usami, R., Horikoshi, K., and Yamaguchi, 1. 2000. Sensitivity to phenylpyrrole fungicides and abnormal glycerol accumulation in os and cut mutant strains of Neurospora crassa. J. Pestic. Sci. 25: 31-36

Non-Patent Document 6: Ochiai, N., Fujimura, M., Motoyama, T., Ichiishi, A., Usami, R., Horikoshi, K., and Yamaguchi, I. 2001. Characterization of mutations in the two-component histidine kinase gene that confer fludioxonil resistance and osmotic sensitivity in the os-1 mutants of Neurospora crassa. Pest Manag. Sci. 57: 437-442.

Non-Patent Document 7: Oshima, M., Fujimura, M., Banno, S., Hashimoto, C., Motoyama, T., Ichiishi, A., and Yamaguchi, 1. 2002. A point mutation in the two-component histidine kinase BcOS-1 gene confers dicarboximide resistance in field isolates of Botrytis cinerea. Phytopathology, 92, 75-80.

Non-Patent Document 8: Alex et al., 1996 Proc. Natl. Acad. Sci. USA 93: 3416-3421.

### DISCLOSURE OF THE INVENTION

An object of the present invention is to provide a method for effectively screening agricultural chemical candidates or drug candidates. Specifically, an object of the present invention as to agricultural chemicals is to effectively screen, without the use of plants, agricultural chemical candidate compounds that do not exert a harmful effect on other organisms such as plants.

The present inventors have conducted diligent studies on a method for screening agricultural chemicals that specifically act on pathogenic microbes causing plant diseases and have consequently considered that agricultural chemicals that specifically inhibit phytopathogenic microbes and do not influence other organisms can be searched by screening agricultural chemical candidates that are targeted for an enzyme present only in the phytopathogenic microbes or for a signal transduction pathway in which the enzyme is involved.

Thus, taking filamentous fungus control as an example, the present inventors have investigated whether an enzyme present only in filamentous fungi or a signal transduction system via the enzyme can be utilized. As described above, the analysis of the sites of action of three kinds of reagent groups (phenylpyrroles, dicarboximides, and aromatic hydrocarbons) (FIG. 1) serving as agricultural chemicals for controlling filamentous fungi has revealed that targets of many filamentous fungus-specific agricultural chemicals including fludioxonil belonging to phenylpyrroles are a signal transduction system via filamentous fungus-specific histidine kinase (Os-1 family). The present inventors have initially developed a method and kit for screening agricultural chemical candidates by utilizing this signal transduction system.

Specifically, the present inventors have completed the present invention by successfully developing a screening method comprising transforming, with an expression vector of the filamentous fungus-specific enzyme-encoding gene, a yeast that is biologically closely related to a filamentous fungus but does not have the enzyme, and applying agricultural chemical candidates samples to a control yeast (which does not express a filamentous fungus-specific enzyme) and to the filamentous fungus-specific enzyme-expressing transformant to select, as an agricultural chemical candidate, an agricultural chemical candidate sample that does not exert an effect such as side-effects on the control yeast and exhibits growth inhibitory or fungicidal activity specific only for the filamentous fungus-specific enzyme-expressing transformant.

This screening method can also be used in the screening of a drug candidate.

The present specification encompasses contents described in the specification and/or drawings of Japanese Patent Application No. 2004-061273 that serves as a basis of the priority of the present application.

### BRIEF DESCRIPTION OF THE DRAWINGS

FIG. 1 shows three kinds of reagent groups targeted for filamentous fungi: the structures of reagents and organisms for which the reagents are used in pathogenic filamentous fungus control;

FIG. 2 is a schematic diagram of bacterial and eukaryotic histidine kinases;

FIG. 3 is a schematic diagram of Os-1 family histidine kinase and Saccharomyces cerevisiae histidine kinase;

FIG. 4 shows the comparison of a signal transduction system between a filamentous fungus and Saccharomyces cerevisiae; a region predicted to be common to Saccharomyces cerevisiae and Neurospora crassa is shaded. Neurospora crassa proteins in parentheses are those having homology to yeast proteins but no confirmed complementation;

FIG. 5 shows a result of the reagent sensitivity test of Saccharomyces cerevisiae introducing rice blast fungus HIK1 therein: A: a plasmid capable of expressing HIK1 under the control of a GAL1 promoter was introduced into a yeast to examine sensitivity to a variety of reagents under expression-inducing conditions; B: a suspension of a yeast cell introducing pYES2-HIK1 therein and a yeast cell (control) introducing pYES2 therein were added (5 μl each) dropwise at 9 mm spacings (from the left, 10⁷, 10⁶, 10⁵, and 10⁴/ml) onto the upper rows and lower rows, respectively, of plates of 90 mm in diameter containing a variety of reagents, followed by culture at 30° C. for 60 hours. Fludioxonil, iprodione, chloroneb, and cycloheximide were added to each SG medium in order from the second leftmost plate. Reagent concentrations are 5 ppm in the uppermost plate and 25 ppm in the second plate for fludioxonil, iprodione, and chloroneb, and 50 ppm in the third plate and 100 ppm in the fourth plate for iprodione and chloroneb. A plate that contained cycloheximide at a concentration of 0.25 ppm was cultured for 240 hours;

FIG. 6 shows dependence of reagent sensitivity conferred by HIK1 on a histidine kinase domain and a response regulator domain: A: a yeast expressing Hik1-H736V having a nonfunctional histidine kinase domain or Hik1-D1153E having a nonfunctional response regulator domain was prepared; B: a suspension of a yeast cell introducing pYES2 therein, a suspension of a yeast cell introducing pYES2-HIK1 therein, a suspension of a yeast cell introducing pYES2-hik1-H736V therein, and a suspension of a yeast cell introducing pYES2-hik1-D1153E therein were added (5 μl each) dropwise at 9 mm spacings (from the left, 10⁷, 10⁶, 10⁵, and 10⁴/ml) onto the uppermost rows, second upper rows, third upper rows, and lowermost rows, respectively, of plates of 90 mm in diameter containing a variety of reagents, followed by culture at 30° C. for 72 hours. The reagents were added to each SG medium so that 25 ppm fludioxonil, 25 ppm iprodione, 50 ppm chloroneb, and 0.5 M NaCl were contained in order from the second leftmost plate;

FIG. 7 shows dependence of reagent sensitivity conferred by HIK1 on SSK1 and HOG1: A: an ssk1 mutant strain and a hog1 mutant strain, even though introducing HIK1 therein, exhibit no reagent sensitivity. A suspension of a control yeast cell introducing pYES2-HIK1 therein (uppermost rows), a suspension of a hog1 mutant strain cell introducing pYES2-HIK1 therein (second rows), a suspension of an ssk1 mutant strain cell introducing pYES2-HIK1 therein (third rows), a suspension of a ste11 mutant strain cell introducing pYES2-HIK1 therein (fourth rows), a suspension of a control yeast cell introducing pYES2 therein (fifth rows), a suspension of a hog1 mutant strain cell introducing pYES2-HIK1 therein (sixth rows), a suspension of an ssk1 mutant strain cell introducing pYES2-HIK1 therein (seventh rows), and a suspension of a ste11 mutant strain cell introducing pYES2-HIK1 therein (lowermost rows) were added (5 μl each) dropwise at 9 mm spacings (from the left, 10⁷, 10⁶, 10⁵, and 10⁴/ml) in order from the uppermost rows of plates of 90 mm in diameter containing a variety of reagents, followed by culture at 30° C. for 60 hours (or 96 hours for NaCl). The reagents were added to each SG medium so that 25 ppm fludioxonil, 25 ppm iprodione, 50 ppm chloroneb, and 0.5 M NaCl were contained in order from the second leftmost plate; B: an ssk1 mutant strain and a hog1 mutant strain, which introduced therein SSK1 and HOG1, respectively, exhibited reagent sensitivity in the presence of HIK1. A suspension of a hog1 mutant strain cell introducing pCLΔ-HOG1 and pYES2-HIK1 therein (uppermost rows), a suspension of a hog1 mutant strain cell introducing pCLΔ and pYES2-HIK1 therein (second rows), a suspension of an ssk1 mutant strain cell introducing pCLΔ-SSK1 and pYES2-HIK1 therein (third rows), and a suspension of an ssk1 mutant strain cell introducing pCLΔ and pYES2-HIK1 therein (lowermost rows) were added (5 μl each) dropwise at 9 mm spacings (from the left, 10 ⁷, 10⁶, 10⁵, and 10⁴/ ml) in order from the uppermost rows of plates of 90 mm in diameter containing a variety of reagents, followed by culture at 30° C. for 60 hours (or 96 hours for NaCl). The reagents were added to each SG medium so that 25 ppm fludioxonil, 25 ppm iprodione, 50 ppm chloroneb, and 0.5 M NaCl were contained in order from the second leftmost plate; and

FIG. 8 shows the interaction between rice blast fungus Hik1 and Saccharomyces cerevisiae Ypd1: A: the general outline of CytoTrap two-hybrid system is shown. The presence of interaction between a target and a bait transfers hSos to a cell membrane to activate Ras, with the result that a cdc25H strain can grow even at 37° C. In this test, whether Hik1 serving as a target “takes” Ypd1 or Ssk1 serving as a bait was investigated; B: the interaction between Hik1 and Ypd1 is shown. Cell suspensions (from the left, 10⁷, 10⁶, 10⁵, and 10⁴/ml) were added (5 μl each) dropwise and cultured for 5 days at each temperature shown.

### BEST MODE FOR CARRYING OUT THE INVENTION

The present invention encompasses a screening method comprising transforming, with an expression vector of a gene encoding an enzyme specific for pathogenic filamentous fungi causing plant or animal diseases, organisms such as other microorganisms that are biologically closely related to the pathogenic microbes but do not have the enzyme, and applying agricultural chemical or drug candidate samples to a control microorganism (e.g., which is derived from the same host and does not express a filamentous fungus-specific enzyme) and to the filamentous fungus-specific enzyme-expressing transformant to select agricultural chemical or drug candidates that do not exert an effect such as side-effects on the control microorganism and exhibits growth inhibitory or fungicidal activity specific only for the filamentous fungus-specific enzyme-expressing transformant. The present invention also encompasses a transformant therefor and a kit therefor.

Pathogenic microbes targeted by the present invention encompass filamentous fungi, for example rice blast fungi for plant diseases and Candida, Aspergillus, athlete's food fungi (Trichophyton) for animal (e.g., human) diseases. Pathogenic filamentous fungus-specific enzymes encompass filamentous fungus-specific histidine kinase (Os-1 family). In the screening of an agricultural chemical or drug targeted for a filamentous fungus, examples of host microorganisms to which a filamentous fungus-specific enzyme gene is introduced can include yeasts, preferably Saccharomyces cerevisiae.

To be more specific, the present invention encompasses a kit comprising a combination of a yeast (expression yeast) that expresses a filamentous fungus-specific histidine kinase gene derived from a filamentous fungus and a yeast (nonexpression yeast) that does not express the gene, and also encompasses a method for screening agricultural chemical candidates using the kit.

[Pathogenic Microbe-Specific Reagent and its Target Enzyme, Taking Plant Disease as Example]

Filamentous fungus-specific agricultural chemicals have previously been known as phytopathogenic microbe-specific agricultural chemicals. Phenylpyrroles (fludioxonil and fenpiclonil), dicarboximides (iprodione and vinclozolin), and aromatic hydrocarbons (chloroneb and PCNB) are known as the filamentous fungus-specific agricultural chemicals.

Recent studies, for example Non-Patent Documents 2 to 7 described above, have revealed that targets of these many filamentous fungus-specific agricultural chemicals are filamentous fungus-specific histidine kinase.

[Histidine Kinase and its Signal Transduction System]

Reversible phosphorylation including the modification of serine, threonine, aspartic acid, histidine, or tyrosine contained in signal proteins is involved in intracellular signal transduction. Histidine kinase is one of signaling factors that exist in bacteria, yeasts, filamentous fungi, and plants.

Prokaryotes have a two-component signal transduction system consisting of two components as basic signaling factors: autophosphorylating histidine kinase and a response regulator that receives phosphoric acid therefrom to send the information to the downstream region.

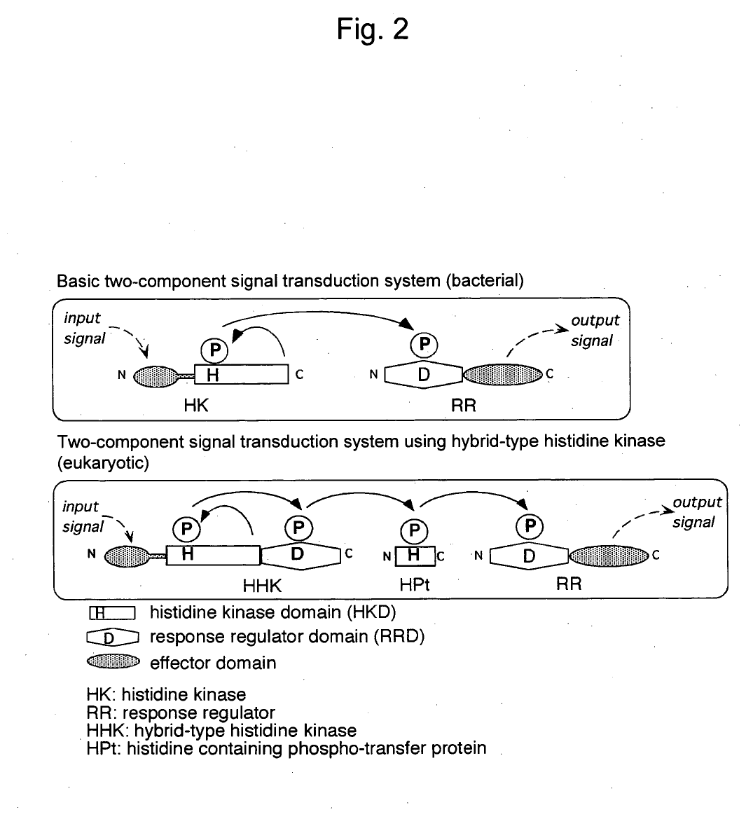

The histidine kinases of eukaryotes are mostly hybrid-type histidine kinases that have both of a histidine kinase domain and a response regulator domain (FIG. 2) (Ota, I. M., and Varshavsky, A. 1993. Science 262: 566-569; Urao et al. 1999. Plant Cell 11: 1743-1754; Pott et al., 2000 Fungal Genet. Biol. 31: 55-67; Virginia et al., 2000 Curr. Genet. 37: 364-372; and West and Stock, 2001 Trends Biochem. Sci. 26: 369-376).

A signal transduction system via eukaryotic histidine kinase consists of three components, hybrid-type histidine kinase, a histidine-containing phospho-transfer protein, and a response regulator.

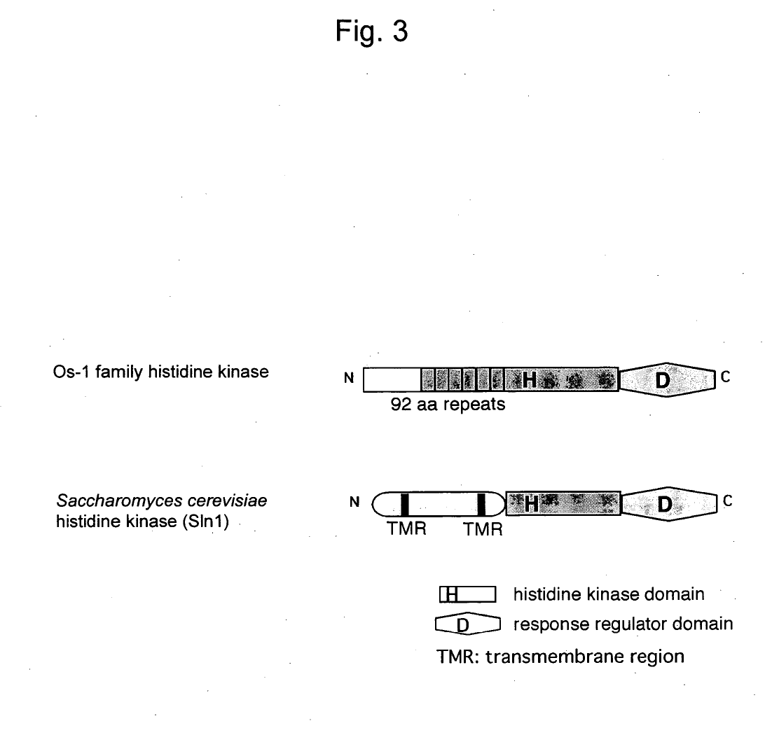

Os-1, which is one of hybrid-type histidine kinases and was found from Neurospora crassa, is characterized by having N-terminal six repeat sequences of 92 to 72 amino acids having homology to each other (FIG. 3) (Alex et al., 1996; and Schumacher et al., 1997). Hybrid-type histidine kinase (Os-1 family) having such a characteristic has been found only from fungi (filamentous fungi) exhibiting mycelial growth such as rice blast fungi (Pyricularia oryzae: Magnaporthe grisea in the perfect stage thereof) and Aspergillus nidulans (Alex et al., 1996 Proc. Natl. Acad. Sci. USA 93: 3416-3421; Schumacher et al., 1997 Curr. Microbiol. 34: 340-347; Alex et al., 1998 Proc. Natl. Acad. Sci. USA 95: 7069-7073; and Nagahashi et al., 1998 Microbiology 144: 425-432).

[Preparation of Filamentous Fungus-Specific Reagent-Sensitive Yeast]

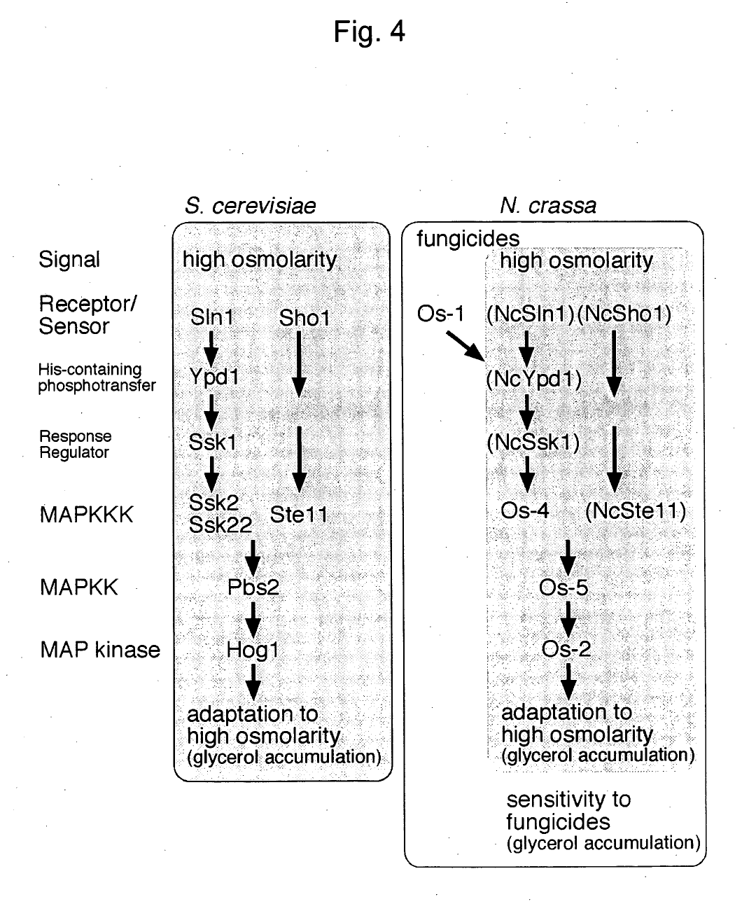

Os-1 family has been found only in filamentous fungi and considered to be an enzyme targeted by filamentous fungus-specific reagents. On the other hand, yeasts that are eukaryotic microorganisms as with filamentous fungi are biologically closely related thereto. Nevertheless, they have no Os-1 family. For example for S. cerevisiae, the full genomic sequence was determined. This yeast has only one histidine kinase, which has a different characteristic (Sln1, see FIG. 3) from that of Os-1 family (Sln1 does not have N-terminal six amino acid repeats and has two transmembrane regions). However, interestingly, most of downstream factors of the signal transduction system of filamentous fungus Os-1 family are in common with those of Saccharomyces cerevisiae (FIG. 4) (Maeda et al., 1995 Science 269: 554-558; Posas et al., 1996 Cell 86: 865-875; and Fujimura et al., 2003 Biosci. Biotechnol. Biochem. 67: 186-191 (2003)).

Thus, a gene of a protein belonging to Os-1 family was introduced into a yeast having no Os-1 family protein gene to prepare a filamentous fungus-specific reagent-sensitive yeast.

Examples of Os-1 family histidine kinase include hybrid-type histidine kinase having a histidine kinase domain and response regulator domain derived from fungi (filamentous fungi) performing mycelial growth, and having a region exhibiting 50% or more homology to the full length of a region containing six amino acid repeats (amino acid repeat 1 (171-260, 90 aa), amino acid repeat 2 (261-352, 92 aa), amino acid repeat 3 (353-444, 92 aa), amino acid repeat 4 (445-536, 92 aa), amino acid repeat 5 (537-628, 92 aa), and amino acid repeat 6 (629-700, 72 aa)) that exist in Neurospora crassa hybrid-type histidine kinase Os-1 (SEQ ID NO: 17) and exhibit homology to each other.

Concrete examples of proteins belonging to Os-1 family can include rice blast fungus Os-1 family histidine kinase Hik1 (DDBJ/EMBL/GenBank accession number AB041647-1), Neurospora crassa Os-1 family histidine kinase Nik-1/Os-1 (Proc. Natl. Acad. Sci. vol. 93, pp. 3416-3421, accession number U50263-1), Gibberella moniliformis Nik1 (accession number AY456038-1), Botryotinia fuckeliana (Botrytis cinerea) Bos1 (Phytopathology, 92, 75-80, accession number AF435964-1), Nectria haematococca Fik (accession number U61838-1), Emericella nidulans (Aspergillus nidulans) Hk4 (accession number AY282750-1), Cochliobolus heterostrophus Bmnhk1 (accession number AB095748-1), Alternaria brassicicola AbNIK1p (accession number AY700092-1), Candida yeast Candida albicans CaNIK1 (accession number AB006363-1), Yarrowia lipolytica CaNIK1 analogous proteins (accession number CR382131-129), and Cryptococcus neoformans putative histidine kinase (accession number AE017343-338).

The present invention can further encompass genes encoding polypeptides that are composed of peptides represented by amino acid sequences encoded by these Os-1 family histidine kinase genes or of peptides represented by the amino acid sequences with the deletion, substitution, and/or addition of some amino acids, and have histidine kinase activities (the histidine kinase domain and the response regulator domain function). In this context, some amino acids mean 1 to 200 amino acids, preferably 1 to 100 amino acids, more preferably 1 to 50 amino acids, even more preferably 1 to 20 amino acids, much more preferably 1 to 9 amino acids.

These Os-1 family histidine kinase genes can also encompass genes having 80% or more homology, preferably 85% or more homology, more preferably 90% homology, even more preferably 95% or more homology, in the comparison thereof with the above-described os-1 family genes under usual default conditions, and encoding polypeptides having histidine kinase activities (the histidine kinase domain and the response regulator domain function).

Preferable examples thereof can include: rice blast fungus-derived HIK1; and a gene encoding a polypeptide that is composed of a peptide represented by the amino acid sequence represented by SEQ ID NO: 16 or of a peptide represented by the amino acid sequence represented by SEQ ID NO: 16 with the deletion, substitution, and/or addition of one to several amino acids, and has histidine kinase activities (the histidine kinase domain and the response regulator domain function).

Examples of organisms to which these Os-1 family histidine kinase genes are introduced include microorganisms and cultured plant cells. Preferably, organisms containing a signal transduction system (which consists of a histidine-containing phospho-transfer protein, response regulator, MAPKKK, MAPKK, and MAP kinase) located downstream of Os-1 are desirable. Concrete examples thereof can include microorganisms endogenously having Ypd1 as the histidine-containing phospho-transfer protein, Ssk1 as the response regulator, and Hog1 as the MAP kinase, preferably yeasts, particularly preferably Saccharomyces cerevisiae or microorganisms belonging to the genus Saccharomyces.

A gene belonging to Os-1 family, for example HIK1, can be recombined into well known expression vectors for desired organisms to which the gene is introduced, and then introduced into the desired organisms. For example, for introduction into yeasts, expression vectors such as pYES2, pYEp51, YEp62, pBM150, pLGDSD5, pAM82, pYE4, pAAh5, pMA56, pAH9/10/21, pMA230, pMA91, and pG-1/2 can be used.

Any of well known means can be used as a method for introducing the recombinant vector to host organisms (desired organisms). For example, a lithium acetate method (Ito et al., 1983 J Bacteriol. 153: 163-168) can be used in the transformation of yeasts.

[Screening Method and a Kit Therefor]

[I] Examples of a kit for screening an agricultural chemical candidate or drug candidate compound of the present invention include

(1) a kit comprising: a transformant prepared by transforming a host organism such as a yeast with an expression vector of a filamentous fungus-specific gene such as an Os-1 family histidine kinase gene; and a control organism of the same host that does not express a filamentous fungus-specific gene,

preferably

(2) (1) a kit comprising: a transformant prepared by transforming a yeast with an expression vector of an Os-1 family histidine kinase gene; and a control yeast prepared by transforming a host yeast with only a vector.

The kit can further contain reagents for measurement, and so on, used in screening described below.

[II] A method for screening an agricultural chemical candidate or drug candidate compound of the present invention comprises the steps of:

(1) administering an agricultural chemical candidate sample or drug candidate sample to a transformant prepared by transforming a host organism such as a yeast with an expression vector of a filamentous fungus-specific gene such as an Os-1 family histidine kinase gene, and to a control organism of the same host that does not express a filamentous fungus-specific gene;

(2) culturing the filamentous fungus-specific gene-expressing transformant and the control organism administered with the agricultural chemical candidate sample or drug candidate sample for a fixed time; and

(3) after the culture for a fixed time, measuring survival rates (or viable cell counts) of the filamentous fungus-specific gene-expressing transformant and the control organism.

Preferably, the screening method comprises the steps of:

(1) administering an agricultural chemical candidate sample to a transformant prepared by transforming a yeast with an expression vector of an Os-1 family histidine kinase gene, and to a control yeast prepared by transforming a host yeast with only a vector;

(2) culturing the Os-1 family histidine kinase gene-expressing transformant and the control yeast administered with the agricultural chemical candidate sample for a fixed time; and

(3) after the culture for a fixed time, measuring growth (or viable cell counts) of the Os-1 family histidine kinase gene-expressing transformant and the control yeast.

Hereinafter, screening of the present invention will be described by taking screening using a transformed yeast as a specific example. However, screening using other microorganisms or organisms can be performed in the same way.

The growth or viable cell counts of the Os-1 family histidine kinase gene-expressing transformed yeast and the control yeast can be measured by, for example visual inspection or OD₆₀₀ measurement or by other appropriate well known methods such as the measurement of enzyme consumptions of yeasts, the measurement of reductions in sugar concentrations in a medium, and measurement using yeast-specific labeled antibodies with labels such as fluorescent or chromogenic enzymes or yeast-specific antibodies labeled with biotin or the like.

(i) Plate method

The transformant from the yeast transformed with the expression vector of an Os-1 family histidine kinase gene and the control yeast are separately diluted to appropriate cell counts, for example 10⁷ cells/ml, 10⁶ cells/ml, 10⁵ cells/ml, and 10⁴ cells/ml. Equal amounts of cell-containing solutions (e.g., 5 μl) are added dropwise to plates (e.g., 90-mm plates) containing fixed amounts of agricultural chemical candidate samples, followed by culture at a culture temperature of 25° C. to 37° C. , preferably 30° C. , for an appropriate time, for example 5 hours or more to 300 hours or less, preferably 48 to 72 hours. The growth states of the Os-1 family histidine kinase gene-expressing transformed yeast ant the control yeast on the plates are confirmed by visual inspection to select, as agricultural chemical candidates or drug candidates, agricultural chemical candidate samples or drug candidate samples that have allowed the growth state (viable count) to differ between the control yeast and the Os-1 family histidine kinase gene-expressing transformant.

(ii) Liquid Culture Method

The transformant from the yeast transformed with the expression vector of an Os-1 family histidine kinase gene and the control yeast are separately cultured for 8 hours to 10 hours, followed by OD₆₀₀ measurement. Next, they are added at, for example OD₆₀₀=0.01, to media containing fixed amounts of agricultural chemical candidate samples or drug candidate samples. After culture at an appropriate temperature, for example 27° C. , preferably shaking culture, and shaking by spinning at 160 rpm, their OD₆₀₀ values are measured at regular time intervals from appropriate elapsed time, preferably at regular time intervals within the time range of ±50% of the doubling time of the control yeast. When an ATCC201388 strain transformed with a vector pYES2 is used as a control yeast, OD₆₀₀ is measured at, for example 3-hour intervals. A growth curve is created from this measurement to calculate a doubling time. Agricultural chemical candidate samples or drug candidate samples that have increased the doubling time of the Os-1 family histidine kinase gene-expressing transformant to 20% or more, preferably 50% or more, more preferably 100 or more, with respect to that of the control yeast are selected as agricultural chemical candidates or drug candidates.

Furthermore, the following method can also be adopted:

(iii) Reagents are administered by placing paper disks soaking the reagents therein onto plates in which each of the yeasts is plated. After static culture at approximately 30° C., growth-inhibited portions are evaluated by visual inspection or the like.

It has previously been suggested that targets of fludioxonil and iprodione are Os-1 family histidine kinase in Candida albicans, a pathogen of candidiasis (Ochiai et al., 2002. Biosci. Biotechnol. Biochem. 66: 2209-2215), Alternaria alternata (Dry et al., 2004. Fungal Genet Biol. 41: 102-108), in addition to Neurospora crassa (Ochiai et al., 2001. Pest Manag Sci. 57: 437-442) and rice blast fungi. Likewise, agricultural chemical candidates or drug candidates screened by the method using Os-1 family histidine kinase derived from some filamentous fungus as a target can usually be agricultural chemical or drug candidates to be investigated for other filamentous fungi having Os-1 family histidine kinase.

Material

Yeast strains and plasmids used are shown in Table 1 below.

TABLE 1
Strains and plasmids

| | genotype | origin |
|---|---|---|
| S. cerevisiae strain | | |
| ATCC201388 | MATa his3Δ1 leu2Δ0 met.j 5Δ0 ura3Δ0 | ATCC* |
| ATCC4002724 | hog1Δ of ATCC201388 | ATCC |
| ATCC4001561 | ssk1Δ of ATCC201388 | ATCC |
| ATCC4005271 | ste1JΔ of ATCC201388 | ATCC |
| ATCC4000993 | slt2Δ of ATCC201388 | ATCC |
| cdc25H | MATa ade2-J01 his3-200 leu2-3 ll2 lys2-80J trpJ-901 | Stratagene |
| | ura3-52 cdc25-2 Gal* | |
| plasmid | | |
| pYES2 | 2 μm URA3 | Invitrogen |
| pYES2-HIK1 | HIK1 in pYES2 | this study |
| pYFS2-hik1-H736V | hik1-H736V in pYES2 | this study |
| pYES2-hik1-D1153E | hik1-D1153E in pYES2 | this study |
| pCLΔ | CEN LEU2 | this study |
| pCLΔ-SSK1 | SSK1 in pCLΔ | this study |
| pCLA-HOG1 | HOG1 in pCLΔ | this study |
| pSos | 2 μm LEU2 | Stratagene |
| pSos-SSK1 | SSK1 in pSos | this study |
| pSos-YPD1 | YPD1 in pSos | this study |
| pMyr | 2 μm URA3 | Stratagene |
| pMyr-HIK1 | HIK1 in pMyr | this study |

*ATCC, American type culture collection

Reagents were obtained from Waco Pure Industries. Medium components used were purchased from Difco unless otherwise stated. General methods were used in gene manipulation (Sambrook et al., 1989 Molecular Cloning: a Laboratory Manual, 2nd ed., Cold Spring Harbor Laboratory Press, Cold Spring Harbor, N.Y.). The culture of yeasts was performed at 30° C. unless otherwise stated. A complete medium used was YPD (1% yeast extract, 2% peptone, 2% glucose), while a minimal medium used was SD (0.67% yeast nitrogen base w/o amino acids, 2% glucose, 1× dropout solution (Clontech)) or SG (0.67% yeast nitrogen base w/o amino acids, 2% galactose, 1% raffinose, 1× dropout solution (Clontech)) excluding nutrition sources necessary for selection. For creating plates, agar was added at a concentration of 2%. The cDNA of a rice blast fungus P-2 strain was prepared in the same way as previously reported (Motoyama et al., 1998 Biosci. Biotech. Biochem. 62: 564-566).

### EXAMPLE 1

### Expression and Analysis of Filamentous Fungus Histidine Kinase in Yeast

(1) The cDNA of a rice blast fungus Os-1 family histidine kinase gene HIK1 (DDBJ/EMBL/GenBank accession number AB041647; SEQ ID NO: 1) was inserted into the BamHI site of an expression vector pYES2 for yeasts to obtain pYES2-HIK1. The pYES2-HIK1 can express the full length of Hik1 under the control of a GAL1 promoter without tags. Culture under expression-inhibiting conditions of the GAL1 promoter was performed with a uracil-free synthetic medium (SD/-Ura) using glucose as a carbon source, and culture under expression-inducing conditions thereof was performed with a uracil-free synthetic medium (SG/-Ura) using galactose as a carbon source. An lithium acetate method (Ito et al., 1983 ibid) was used in the transformation of yeasts.

Reagent sensitivity was analyzed by plate culture and liquid culture. In the plate culture, strains were precultured overnight in 5 ml of SD/-Ura and then collected, followed by washing with 10 ml of SG/-Ura. The resulting strains were cultured for 8 hours to 10 hours in 10 ml of SG/-Ura, followed by OD₆₀₀ measurement. They were diluted to 10⁷ cells/ml, 10⁶ cells/ml, 10⁵ cells/ml, and 10⁴ cells/ml, and 5 μl each thereof was added dropwise onto SG/-Ura plates supplemented with a variety of reagents, and cultured for 60 hours to 240 hours. In the liquid culture, strains were precultured overnight in 5 ml of SD/-Ura and then collected, followed by washing with 10 ml of SG/-Ura. The resulting strains were cultured for 8 hours to 10 hours in 10 ml of SG/-Ura, followed by OD₆₀₀ measurement. They were added at OD₆₀₀=0.01 to 300-ml Erlenmeyer flasks accommodating 50 ml of SG/-Ura containing a variety of reagents. After shaking by spinning at 160 rpm and 27° C., sampling was performed after 12, 15, 18, 21, 24, 33, 36, and 39 hours, followed by OD₆₀₀ measurement. A growth curve was created to calculate a doubling time.

(Result)

Imparting of Reagent Sensitivity by Expression of Os-1 Family Histidine Kinase Gene HIK1 in Yeast

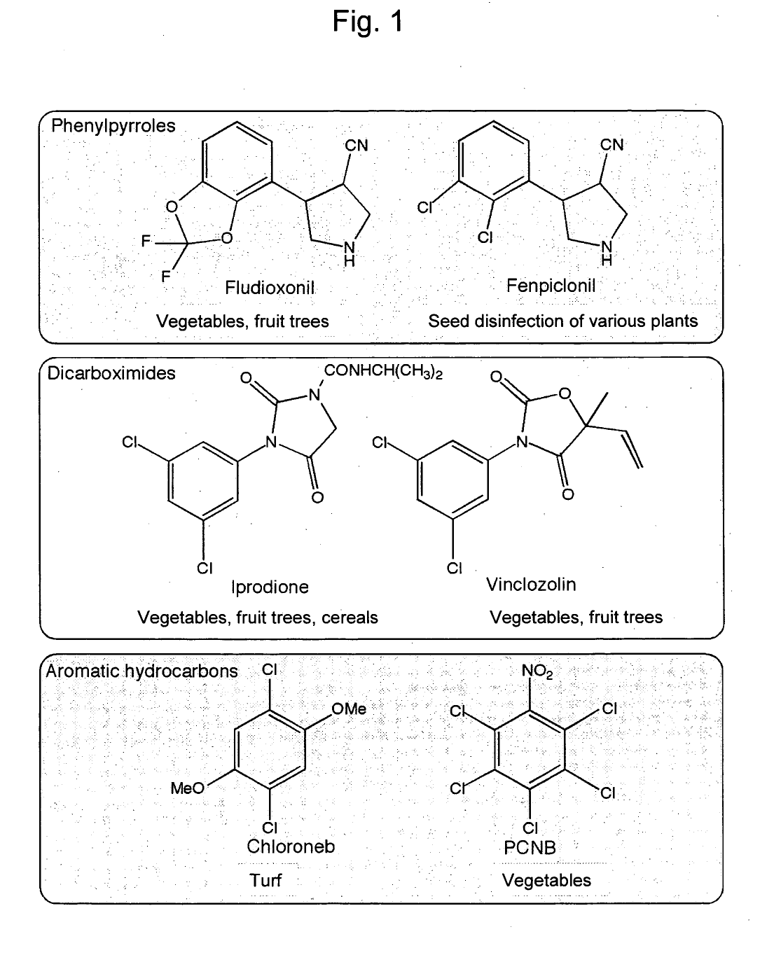

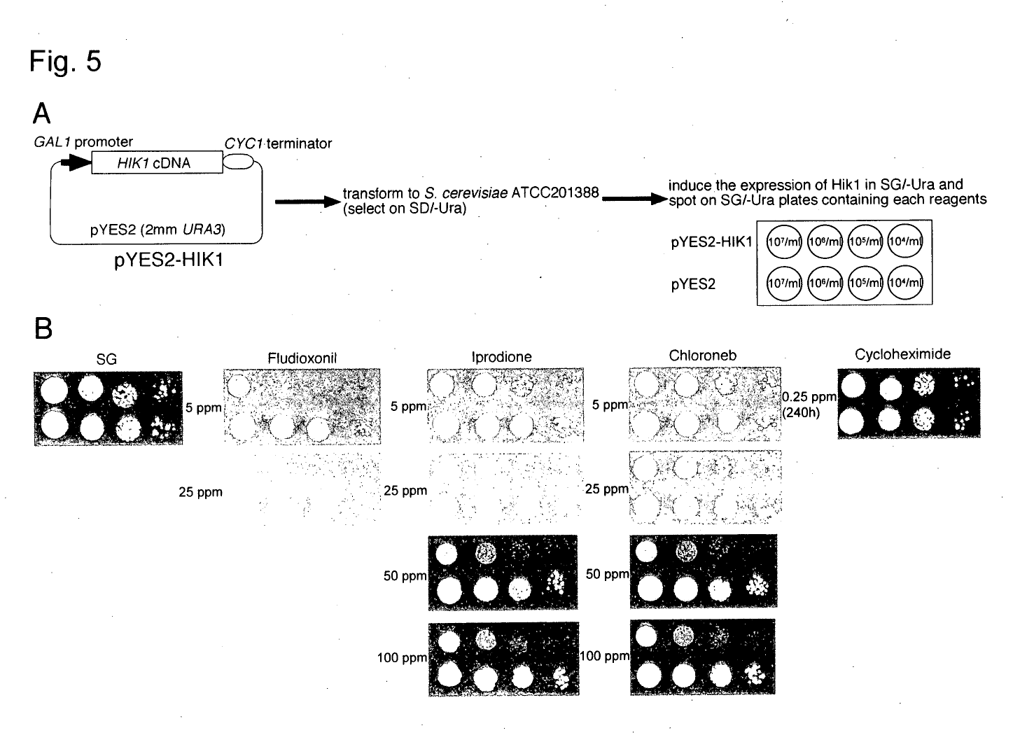

Rice blast fungus-derived Os-1 family histidine kinase Hik1 was expressed under the control of the GAL1 promoter in S. cerevisiae having no Os-1 family histidine kinase in the genome by introducing the pYES2-HIK1 thereinto (FIG. 5A). The pYES2-HIK1 transformant exhibited growth similar to that of a pYES2 transformant in the absence of the reagents under expression-inducing conditions of the GAL1 promoter, while only the pYES2-HIK1 transformant exhibited growth inhibition in the presence of the agricultural chemicals (fludioxonil, iprodione, and chloroneb, see FIG. 1) specific to filamentous fungi originally exhibiting no sensitivity (FIG. 5B). The effects on fludioxonil, iprodione, and chloroneb were saturated at the concentrations of 5 ppm, 25 ppm, and 50 ppm, respectively. Moreover, even the pYES2-HIK1 transformant did not exhibit growth inhibition under expression-inhibiting conditions of the GAL1 promoter (data not shown). No difference in sensitivity to cycloheximide structurally different from these three types of reagent groups was observed between the pYES2-HIK1 transformant and the pYES2 transformant even under expression-inducing conditions. As described above, sensitivity to these three types of reagents was imparted to the pYES2-HIK1 transformant in a manner specific only for the expression-inducing conditions of the GAL1 promoter. Therefore, this sensitivity was considered to be specifically caused by the introduced HIK1.

When a similar experiment was performed by liquid culture (Table 2), the pYES2 transformant exhibited a similar growth rate under expression-inducing conditions of the GAL1 promoter, regardless of the presence or absence of the reagents (0 ppm or 25 ppm fludioxonil, 25 ppm iprodione, and 25 ppm chloroneb), and its doubling time became around 2.2 hours. The pYES2-HIK1 transformant was influenced by the reagents, and its doubling time was 2.19 hours, which was almost the same as the doubling time of the pYES2 transformant, in the absence of the drugs, and however, was 4.25 hours in the presence of 25 ppm fludioxonil, 3.12 hours in the presence of 25 ppm iprodione, and 2.90 hours in the presence of 25 ppm chloroneb, showing evident reductions in growth rate.

TABLE 2
Rice blast fungus HIK1 imparts reagent sensitivity to
Saccharomyces cerevisiae

| yeast strain | reagent | doubling time* |
|---|---|---|
| ATCC201388 [pYES2] | — | 2.17 ± 0.05 |
| ATCC201388 [pYES2] | 25 ppm Fludioxonil | 2.25 ± 0.06 |
| ATCC201388 [pYES2] | 25 ppm Iprodione | 2.12 ± 0.06 |
| ATCC201388 [pYES2] | 25 ppm Chloroneb | 2.17 ± 0.08 |
| ATCC201388 [pYES2-HIK1] | — | 2.19 ± 0.06 |
| ATCC201388 [pYES2-HIK1] | 25 ppm Fludioxonil | 4.25 ± 0.20 |
| ATCC201388 [pYES2-HIK1] | 25 ppm Iprodione | 3.12 ± 0.10 |
| ATCC201388 [pYES2-HIK1] | 25 ppm Chloroneb | 2.90 ± 0.11 |

*average ± standard deviation

(2) Mutagenized HIK1 was prepared with Mutan-Express Km kit (TAKARA SHUZO) using synthetic DNAs for mutagenesis (HK-H736V: 5′-CCTCGCTAACATGTCCGTCGAAATCCGCACACC-3′ (SEQ ID NO: 2) and HK-D1153E: 5′-GATGTGATCCTGATGGAGGTTCAAATGCCTGTCATG-3′ (SEQ ID NO: 3)). Each mutagenized HIK1 was cloned into the BamHI site of pYES2 to prepare pYES2-hik1-H736V and pYES2-hik1-D11153E.

(Result)

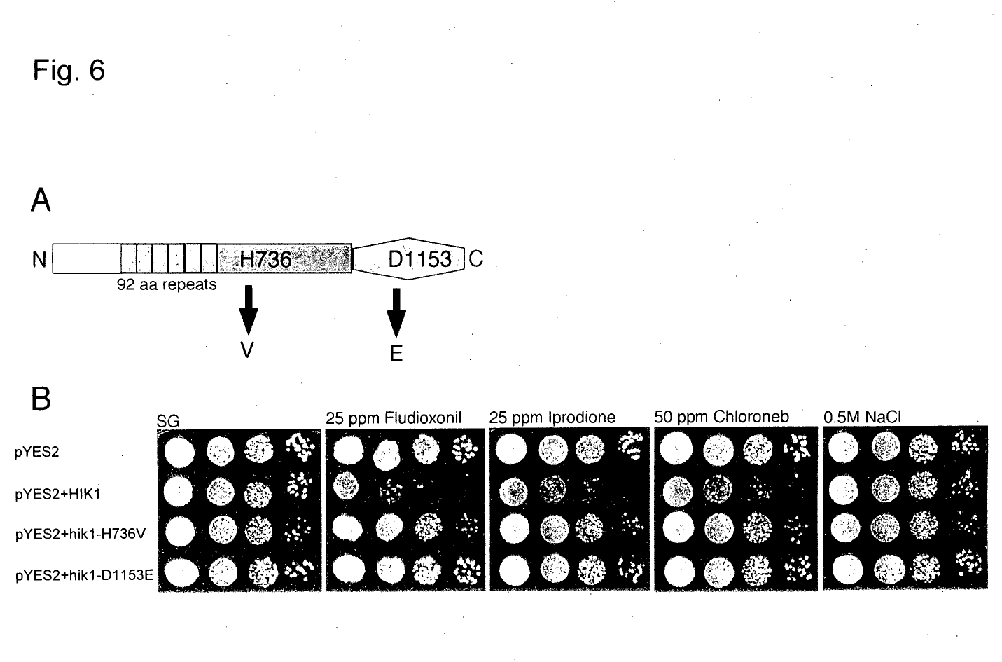

To investigate whether domains necessary for the function of histidine kinase were involved in the imparting of reagent sensitivity by Hik1, a plasmid pYES2-hik1-H736V for expressing Hik1-H736V where autophosphorylated H736 necessary for the function of the histidine kinase domain was mutagenized to make the domain nonfunctional, and a plasmid pYES2-hik1-D1153E for expressing Hik1-D1153E where D1153 receiving phosphoric acid in the phospho-relay of the response regulator domain was mutagenized to make the domain nonfunctional were prepared and introduced into ATCC201388 strains in the same way (FIG. 6A). Unlike the strain introducing pYES2-HIK1 therein, both of the strain introducing pYES2-hik1-H736V therein and the strain introducing pYES2-hik1-D1153E therein hardly exhibited sensitivity to three types of reagents (25 ppm fludioxonil, 25 ppm iprodione, and 50 ppm chloroneb) (FIG. 6B), suggesting that both of the histidine kinase domain and the response regulator domain are required for imparting reagent sensitivity. Slight growth inhibitory effects were observed in the strain introducing pYES2-hik1-H736V therein, regardless of the presence or absence of the reagents. Moreover, no particular change was observed in sensitivity to osmotic pressure (0.5 M NaCl).

### REFERENCE EXAMPLE 1

A pathway via yeast Hog1 MAPK is required for the imparting of reagent sensitivity by Hik1.

(1) In filamentous fungi, a signal transduction system via a homolog (Os-2 in Neurospora crassa (Zhang et al., 2002 Appl. Environ. Microbiol. 68:532-538) and Osm1 in rice blast fungi (Dixon et al., 1999 Plant Cell 11: 2045-2058)) for the Hog1 MAP kinase of Saccharomyces cerevisiae works downstream of Os-1 family histidine kinase (FIG. 4).

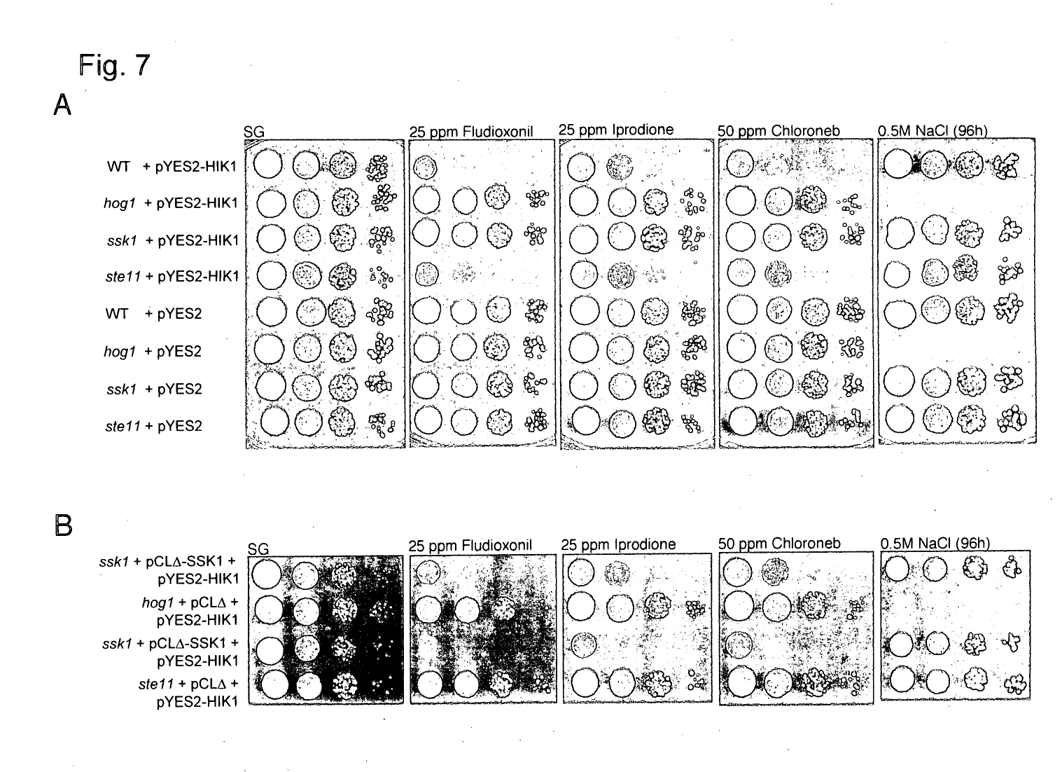

The most highly possible interpretation of the result showing the reagent sensitivity imparted by expressing Hik1 in Saccharomyces cerevisiae is that the signal transduction system to Hog1 MAP kinase of Saccharomyces cerevisiae is disturbed by the action of the reagents on Hik1, causing growth inhibition. To prove this possibility, mutant strains of the factors of this signal transduction system, that is, a hog1 mutant strain, ssk1 mutant strain, and ste11 mutant strain, were used to investigate whether reagent sensitivity was exhibited when pYES2-HIK1 were introduced in each of the mutant strains to induce expression (FIG. 7A). Even though pYES2-HIK1 was introduced into the hog1 mutant strain and the ssk1 mutant strain to induce expression, these mutant strains did not exhibit sensitivity to the reagents (25 ppm fludioxonil, 25 ppm iprodione, and 50 ppm chloroneb). On the other hand, when pYES2-HIK1 was introduced into the ste11 mutant strain to induce expression, it exhibited sensitivity to the reagents (25 ppm fludioxonil, 25 ppm iprodione, and 50 ppm chloroneb), as with wild-type strains. All the strains introducing pYES2 therein exhibited no sensitivity to the reagents, suggesting that this reagent sensitivity is specific to HIK1. The expression of Hik1 did not influence sensitivity to osmotic pressure (0.5 M NaCl). These results suggest that Ssk1 and Hog1 are required for the imparting of reagent sensitivity while Ste11 is unnecessary.

(2) At this stage, the possibility can not be denied that the absence of reagent sensitivity exhibited by introducing pYES2-HIK1 into the hog1 mutant strain and the ssk1 mutant strain to induce expression was due to mutation unrelated to hog1 mutation and ssk1 mutation. Thus, an experiment was performed to determine whether reagent sensitivity was recovered when an intact HOG1 gene and SSK1 gene were introduced into the hog1 mutant strain and the ssk1 mutant strain, respectively, in other words, whether hog1 mutation and ssk1 mutation were responsible for the absence of exhibited reagent sensitivity (FIG. 7B).

Plasmids pCLΔ-HOG1 and pCLΔ-SSK1 for complementing the mutation of yeasts were prepared by cloning ATCC201388 strain-derived HOG1 or SSK1 into the HindIII site of PCLΔ prepared by digesting pCL1 (Clontech) with HindIII and self-ligating it. The HOG1 was amplified with 5′-TTTAAGCTTATCGATTGAAGGAAATAAGAGGAATAGC-3′ (SEQ ID NO: 8) and 5′-TTTAAGCTTGGGTGAGACAGCTATTTAGCAAGTTC-3′ (SEQ ID NO: 9), and the SSK1 was amplified with 5′-TTTAAGCTTCCCACTGCTGGATCGACCATTC-3′ (SEQ ID NO: 10) and 5′-TTTAAGCTTTAGTTGCCAGTCAAGATTTCCC-3′ (SEQ ID NO: 11). DNA sequencing was used to confirm that the amplified genes had no mutation.

When pCLΔ-HOG1 was introduced into the hog1 mutant strain to express a HOG1 gene, sensitivity to the reagents was recovered. Likewise, when pCLΔ-SSK1 was introduced into the ssk1 mutant strain to express a HOG1 gene, sensitivity to the reagents was recovered. In controls for both mutant strains introducing only a vector (PCLΔ) therein, the recovery of reagent sensitivity was not observed.

These results demonstrated that among the factors of the signal transduction system via Hog1 MAPK, Ssk1 and Hog1 are required for the imparting of reagent sensitivity by Hik1 while Ste11 is unnecessary.

### REFERENCE EXAMPLE 2

Analysis of Interaction Between Hik1 and Ypd1

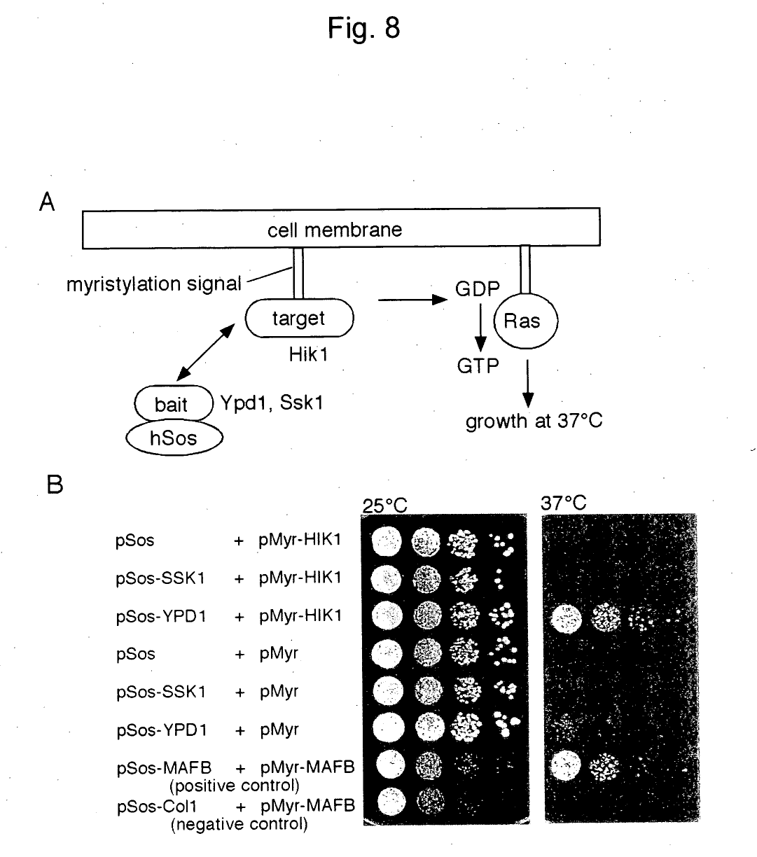

CytoTrap XR library construction kit (Stratagene) was used to analyze protein-protein interaction on the basis of CytoTrap two-hybrid system. This system consists of a vector pSos for expressing one protein as a fusion protein with human Sos, a vector pMyr for expressing the other protein as a fusion protein with a myristylation signal, and a yeast strain cdc25H containing temperature-sensitive mutation in a cdc25 gene, a yeast homolog for Sos. The presence of interaction between two proteins transfers human Sos to a membrane by a myristic acid-modified site to complement the cdc25 mutation of the yeast, with the result that the strain can grow even at a high temperature (37° C. ) (FIG. 8A). *pMyr-HIK1 was constructed by cloning HIK1 cDNA into the SmaI site of pMyr. pSos-YPD1 was constructed by ligating ATCC201388 strain-derived YPD1 digested with SmaI and XhoI to pSos digested with SrfI and SalI. pSos-Ssk1 was constructed by ligating ATCC201388 strain-derived SSK1 digested with SmaI and XhoI to pSos digested with SrfI and SalI. The YPD1 was amplified with 5′-TTTCCCGGGATATGTCTACTATTCCCTCAGAAATC-3′ (SEQ ID NO: 12) and 5′-TTTCTCGAGTTATAGGTTTGTGTTGTAATATTTAGAT-3′ (SEQ ID NO: 13), and the SSK1 was amplified with 5′-TTTCCCGGGATATGCTCAATTCTGCGTTACTGTGG-3′ (SEQ ID NO: 14) and 5′-TTTCTCGAGTCACAATTCTATTTGAGTGGGCG-3′ (SEQ ID NO: 15). DNA sequencing was used to confirm that the amplified genes had no mutation. Procedures from the transformation of yeasts to the dropwise addition of the transformed yeasts were performed in the same way as in the analysis of drug sensitivity described above. However, culture was performed at 25° C. before the dropwise addition and at 25° C. and 37° C. after the dropwise addition.

(Result)

Hik1 Interacts With Signaling Factor Ypd1 Located Upstream of Hog1 of Yeast

To elucidate the site of action of Hik1 in yeasts, the interaction between Hik1 and an interacting factor candidate Ypd1 or Ssk1 of a yeast was analyzed with a yeast two-hybrid system (see FIG. 8A for materials and procedures). A S. Cerevisiae cdc25H strain used in this system can not grow at a high temperature (37° C. ) that can not activate a Ras pathway, due to the temperature-sensitive mutation of a CDC25 gene, and can grow at the allowable temperature of 25° C. A target is expressed as a fusion protein with a myristylation signal, and a bait is expressed as a fusion protein with human Sos (homolog for yeast Cdc25). The presence of interaction between the target and the bait transfers the human Sos to a cell membrane by the myristylation signal to activate the Ras pathway, with the result that the cdc25H strain can grow even at 37° C. As a result, the interaction can be detected.

In this test, whether rice blast fungus Hik1 (expressed as pMyr-HIK1) as a target bonded to Ypd1 (expressed as pSos-YPD1) and Ssk1 (expressed as pSos-SSK1) as baits was analyzed (FIG. 8B). At the allowable temperature of 25° C., growth was observed in all the combinations. However, slight growth inhibition was observed in the combination of positive controls (pSos-MAFB+pMyr-MAFB) exhibiting interaction and the combination of negative controls (pSos-Col1+pMyr-MAFB) exhibiting no interaction. At 37° C., growth was observed in the combination of the positive controls and was not observed in the combination of the negative controls, demonstrating that the experimental system had no problem. Hik1 exhibited interaction with Ypd1 (pSos-YPD1+pMyr-HIK1) and did not exhibit interaction with Ssk1 (pSos-SSK1+pMyr-HIK1). Slight growth at 37° C. was also observed in Ypd1 alone (pSos-YPD1+pMyr). This may be due to interaction with membrane-bound region-containing Sln1 (FIGS. 3 and 4), an interacting partner of Ypd1 in normal yeasts. These results indicated that rice blast fungus Hik1 in Saccharomyces cerevisiae is highly likely to impart reagent sensitivity via the Ypd1 of the Saccharomyces cerevisiae.

### INDUSTRIAL APPLICABILITY

According to the present invention, a candidate of a reagent specifically acting on a filamentous fungus can be obtained selectively in a way impracticable for conventional methods by using a filamentous fungus-specific enzyme as a target. Furthermore, a screening method and kit of the present invention use, in the screening, a yeast that belongs to the same fungi and is closely related to filamentous fungi. Therefore, the method and kit of the present invention are capable of exclusion of a reagent also acting (exerting a side-effect) on the yeast, simultaneously with the selection of a reagent candidate, and produce an excellent effect that can drastically reduce expense and work required for the development of agricultural chemicals and drugs.

The present invention can be utilized in technical fields of agricultural chemical development and drug development.

All publications, patents and patent applications cited herein are incorporated herein by reference in their entirety.

1. A yeast transformed with an Os-1 family histidine kinase gene of a filamentous fungus.

2. The transformed yeast according to claim 1, wherein the Os-1 family histidine kinase gene of a filamentous fungus is a HIK1 gene.

3. The transformed yeast according to claim 1 or 2, wherein the yeast is Saccharomyces cerevisiae.

4. A kit for screening a filamentous fungus-specific agricultural chemical candidate or drug candidate comprising: (1) a yeast transformed with an expression vector of a filamentous fungus-derived Os-1 family histidine kinase gene; and (2) a control yeast (which does not express a filamentous fungus-specific enzyme).

5. The kit for screening a filamentous fungus-specific agricultural chemical candidate or drug candidate according to claim 4, wherein the filamentous fungus-derived Os-1 family histidine kinase gene is a HIK1 gene.

6. The kit for screening a filamentous fungus-specific agricultural chemical candidate or drug candidate according to claim 4 or 5, wherein the yeast is Saccharomyces cerevisiae.

7. A method for screening a filamentous fungus-specific agricultural chemical candidate or filamentous fungus-specific drug candidate comprising the following steps (1) to (3) of:
(1) administering an agricultural chemical candidate sample or drug candidate sample to a yeast transformed with a recombinant expression vector of a filamentous fungus-derived Os-1 family histidine kinase gene and to a control yeast (which does not express an Os-1 family histidine kinase);
(2) culturing the Os-1 family histidine kinase-expressing transformant and the control yeast administered with the agricultural chemical candidate sample or drug candidate sample for a fixed time; and
(3) after the culture for a fixed time, measuring growth rates or viable cell counts of the Os-1 family histidine kinase-expressing transformant and the control yeast.

8. The method according to claim 7, wherein the measurement of growth rates or viable cell counts of the transformant and the control yeast is performed by measuring OD values of culture solutions of the Os-1 family histidine kinase-expressing transformant and the control yeast.

9. The method according to claim 7, wherein the measurement of growth rates or viable cell counts of the Os-1 family histidine kinase-expressing transformant and the control yeast is performed by using a yeast-specific antibody.

10. The screening method according to any one of claims 7 to 9, wherein the filamentous fungus-derived Os-1 family histidine kinase gene is a HIK1 gene.

11. The screening method according to any one of claims 7 to 10, wherein the yeast is Saccharomyces cerevisiae.


SEQUENCE LISTING (17 sequences)

SEQ ID NO: 1 - DNA, 5457 bp, Magnaporthe grisea
taattttccatccccctcgtgtcgccttcgcgcctacttaccgacttaccgccttctcgctgattgagccacggagaggcaattgggcttgggggcgctattttttattttcttattactattctttttttttgaatctttaccaaccttccttggctggtttacttcctcgtctttacactgcaacagtagcacgccatccgaccagcgacaacaatctccaaccctccccagagccacccaatcaaccaacaagcttggttctccacgaccacagcaatcctttgattccctgtcgcgcccgctaacctcatgaattcctaagccgacctggatcaatccaacgcctgcttcggtgtttagggcagctgccgactttttttttcttctacatatatattttcaaatcgtcctatatttgaccgtcggccgagttccgacacccgccacgcgcatcatggcggacgcggcgactctggcagctgtcgctgcgattgtggagaatatcgctaccaactcgggggcccctggaaaaaatgcttcatttcgctccagtacctatgtccagcttcccggtccggaatccgacgagaagaaacagctcgagcgcgagcttgccgccctggtgataagggtacagcagctcgaaacccgtgccaacgcggctcctgctacaatattccccgacacacccaacgaaactgcacattcactctttggcgatgatagctcgtcccctaccagttcgagctcaggccgggagcctaaacgactgaagtcggcatccagcacaacgaggaatggtttcactacggacggtcgtccatcaaagctcaacgcaatcaccgatgaggagctcgaaggcttgcgcgaacatgttgacggccagtcccggctgctcgacagccaaagggccgagctggacggcgtcaatgcccaactcttggagcagaagcagctgcaagagcgcgcccttgccataatcgagcaggaacgtgtagccactttggagagagagctatggaaacatcaaaaggccaacgaggccttccagaaggctctccgggagattggatcgatagtgaccgctgcagcccggggtgacctctctaagagggtcaagataaacccgattgagatggaccctgaaatcaccacattcaagaggaccatgaacgccatgatggatcaacttggcgtcttctctagtgaagtctcgcgagtggcaagagaggtcggcaccgagggcatattaggtggacaggcccagatcgagggagtggacggcacgtggaaagaactgacggacaatggtacggtcgaaacgctgctcatcacctctcctatccataactaccaccgcagatgctaacattgatactttcatacagtcaacgtcatggcgcagaacctgaccgaccaagtccgcgaaatcgcctcagtcactacagctgtggcccacggagatttgacccaaaagattgagagtgcggccaagggagaaatcctacagcttcaacaaactataaataccatggtggaccaactacgcacatttgcttcagaggttacccgtgtcgcccgtgacgtcggaaccgagggaatgctcggcgggcaggctgacgttgaaggggtcaagggcatgtggaatgagctgacggtcaacgtcaacgccatggccaacaatttaacaacccaagtgcgcgacatcatcaacgttaccacagccgtcgcaaagggagatcttacacaaaaggtgcaggcggaatgtcgcggcgagatttttgagctcaagaacacgatcaattccatggtggaccagctgcagcaatttgctcgcgaggttaccaagatcgccagagaggttggtaccgaaggacggctgggcggccaagcaactgttcacgatgtacagggaacttggcgagatctcacagaaaacgtgaacggaatggctatgaatctcaccacacaagtacgagagatagccaatgttaccagtgccgtcgctgcaggcgacctatccaagaagatcagggtagaggtcaagggcgagattctggacctcaaaaataccatcaacaccatggttgaccgcctcggaactttcgccttcgaagtcagcaaagtagcccgagccgtcggcacagatggcactcttggtggtcaggctcaagttgagaatgtggagggcaaatggaaagacctcaccgaaaacgtcaacaccatggcgtcaaacctcacttctcaggtaagcggaccttatccactggattggactggtggcttttcctctgaattcagccctattgtaaatcaatgtatgcaccagtgtgcatgttctgcagggcctgctgtgtgtgcgtcgccagctgttttggagacgctgggcgcatcccggcgtgcgcttgcattttgtcaaccaaatttgtctgcacattgatgcatagcgagcacgtgctaatttttggccgggtcttataggtcaggggaatatcaaccgtgacacaagccatcgcgaacggtgacatgagccgaaagatcgacgtggaagccaagggcgagatactaatcctcaaggaaactatcaacaacatggttgatcgtctgtcgatattctgcaatgaagtacaacgagtcgcaaaagatgtaggcgttgatggcattatggggggacaagccgacgttgcaggtctcaaggggcgatggaaggagattaccaccgatgtcaacaccatggccaacaatcttgtaagtgctggaagatctcaaacaacgggaaactcaagccagtgctaacctaatccgcagacggcgcaagtacgcgctttcggagatataaccaatgccgctaccgacggagacttcaccaagctggtcgaggttgaggcgtcgggcgaaatggacgaactgaagcgcaagatcaatcaaatggtctacaatctccgagacagtatccaaagaaacacgcaagcaagagaagccgcagaattggccaacaagacgaagtcggagttcctcgctaacatgtcccacgaaatccgcacacccatgaacggtatcatcggcatgacacaacttactcttgatacagatttgacgcaataccaacgcgaaatgctcaacattgtcaacaatctcgccatgagtctgctcaccattatcgacgacatcctcgatctgtcaaagattgaggctaagcggatggttatcgaggagattccatacacgttacgaggaacggtcttcaacgcactgaagactttggcggtcaaggcgaacgacaagtttttggatctcacgtaccgtgtggacagctcagttcctgaccacgtcatcggtgactcgttccgtctgcgccagattatcctgaacctggttggcaatgccatcaaattcaccgagcatggagaggtcagccttactatccagaagggcaacgacgtgacgtgcctgccaaacgagtacatgatcgaatttgtcgtgtcggacacgggcataggaattccaacggacaaactgggtctcatcttcgacacattccagcaggctgatggatccatgacacgcaagtttggcggaaccgggcttggtctgtctatttccaagaggctcgtcaacctcatgggcggtgacgtgtgggtcaagtcacaatacggcaagggcagctcgttctacttcacttgtcgtgtccgcctcgccgacgtggatatctcactcatcaggaagcagctgaagccttacaagggacaccaggtcctgttcatcgataagggcaagactggacacgggcccgaggtggggcagatgctcggccagctgggtttggtgcccatcgtgctggaatccgagcaaaatcacaccctgacgcgggtgcgcggcaaggaatgtccctacgacgtgatagttgtcgactcaatcgacacagcccggcgcctgagaggaattgacgacttcaagtatctgcccatcgttctcctggcgccaactgtccacgtcagcctgaaatcctgcttggacttgggtattacctcgtatatgacgatgccctgcaagctcatcgacctcggcaatggtatggttcccgctcttgagaaccgtgccacaccatcactatcagacaacactaagtcgttcgaaattctgctggccgaggacaacaccgtcaaccagcgcctggccgttaagattcttgaaaagtacaaccacgttgtgacggtagtcagcaacggtgctgaagctcttgaagctgtcaaggataacaaatacgatgtgatcctgatggatgttcaaatgcctgtcatggtaagttgatactccctcgtacatattccatgatcctccgttcccgacccgccagatagtctcgataagttccaatactaatacgttgcaacattaatagggtggatttgaggcgacggcaaagattcgtgaatacgagcgcagcctgggcacacagaggacaccaatcatcgcgcttaccgctcacgcaatgatgggcgaccgtgagaagtgtatcgaggcccagatggacgagtacctgtcgaagcctctgcagcagaaccacttgatacaaacaattctcaagtgtgcaacgctgggtggcgccttgttggaacaaaatcgtgagcgcgagcttgaactagcaaggcatgccgaacacaaaggaggactgtctacggacccggcgagggcatcgtcggtaatgcgtccgccactacaccaccgaccggtgactacagccgagtcgctttctggtggcgccgaaagcccctcgttgatggcaaatgacggcgaagatccaatacaaagggcacgtagcagtctctctgaaccaggatgcctataaggctgacagctctggcctcctcgcacttgagggcgagcctgaacatttgtagcttctcttacgatccttgagcgcatagatcactgctgcctttttgtaacagccagcgcgatgacgatgttttcaacagcctatacttttacctataccacaaacgcatacgattatcccggtgtacttctgtctattccctaggagtctgggaggttacattttgttctagtcattaatgggtcgatggccaactggttttggccacaacgcatcaaaaccaaaccagttctgtcatcactgaccttttgttccatgtcggtaatgtcttcattaatattgttacttttgcgggtctgggtctgcttttacgatggatacatcgggggaacaatttctttcttcttgttttgttggatatttgtggtagtttctagatatctgtcgaactacgggaaggtgtggagagtgcattaagaggcggcgcagttgttgtgatcttgacaccccgagatgaccacagtcaacccaatgaataacacaaaatatacaactctctatgtcagtagaccaaatacgtattcgttgtgttagtgtttagcgcaagggaaacc

SEQ ID NO: 2 - DNA, 33 bp, Artificial Sequence
  Synthetic DNA
cctcgctaacatgtccgtcgaaatccgcacacc

SEQ ID NO: 3 - DNA, 36 bp, Artificial Sequence
  Synthetic DNA
gatgtgatcctgatggaggttcaaatgcctgtcatg

SEQ ID NO: 4 - DNA, 37 bp, Artificial Sequence
  Synthetic DNA
tttaagcttatcgattgaaggaaataagaggaatagc

SEQ ID NO: 5 - DNA, 35 bp, Artificial Sequence
  Synthetic DNA
tttaagcttgggtgagacagctatttagcaagttc

SEQ ID NO: 6 - DNA, 31 bp, Artificial Sequence
  Synthetic DNA
tttaagcttcccactgctggatcgaccattc

SEQ ID NO: 7 - DNA, 31 bp, Artificial Sequence
  Synthetic DNA
tttaagctttagttgccagtcaagatttccc

SEQ ID NO: 8 - DNA, 37 bp, Artificial Sequence; identical to SEQ ID NO: 4
  Synthetic DNA

SEQ ID NO: 9 - DNA, 35 bp, Artificial Sequence; identical to SEQ ID NO: 5
  Synthetic DNA

SEQ ID NO: 10 - DNA, 31 bp, Artificial Sequence; identical to SEQ ID NO: 6
  Synthetic DNA

SEQ ID NO: 11 - DNA, 31 bp, Artificial Sequence; identical to SEQ ID NO: 7
  Synthetic DNA

SEQ ID NO: 12 - DNA, 35 bp, Artificial Sequence
  Synthetic DNA
tttcccgggatatgtctactattccctcagaaatc

SEQ ID NO: 13 - DNA, 37 bp, Artificial Sequence
  Synthetic DNA
tttctcgagttataggtttgtgttgtaatatttagat

SEQ ID NO: 14 - DNA, 35 bp, Artificial Sequence
  Synthetic DNA
tttcccgggatatgctcaattctgcgttactgtgg

SEQ ID NO: 15 - DNA, 32 bp, Artificial Sequence
  Synthetic DNA
tttctcgagtcacaattctatttgagtgggcg

SEQ ID NO: 16 - protein, 1307 aa, Magnaporthe grisea
MADAATLAAVAAIVENIATNSGAPGKNASFRSSTYVQLPGPESDEKKQLERELAALVIRVQQLETRANAAPATIFPDTPNETAHSLFGDDSSSPTSSSSGREPKRLKSASSTTRNGFTTDGRPSKLNAITDEELEGLREHVDGQSRLLDSQRAELDGVNAQLLEQKQLQERALAIIEQERVATLERELWKHQKANEAFQKALREIGSIVTAAARGDLSKRVKINPIEMDPEITTFKRTMNAMMDQLGVFSSEVSRVAREVGTEGILGGQAQIEGVDGTWKELTDNVNVMAQNLTDQVREIASVTTAVAHGDLTQKIESAAKGEILQLQQTINTMVDQLRTFASEVTRVARDVGTEGMLGGQADVEGVKGMWNELTVNVNAMANNLTTQVRDIINVTTAVAKGDLTQKVQAECRGEIFELKNTINSMVDQLQQFAREVTKIAREVGTEGRLGGQATVHDVQGTWRDLTENVNGMAMNLTTQVREIANVTSAVAAGDLSKKIRVEVKGEILDLKNTINTMVDRLGTFAFEVSKVARAVGTDGTLGGQAQVENVEGKWKDLTENVNTMASNLTSQVRGISTVTQAIANGDMSRKIDVEAKGEILILKETINNMVDRLSIFCNEVQRVAKDVGVDGIMGGQADVAGLKGRWKEITTDVNTMANNLTAQVRAFGDITNAATDGDFTKLVEVEASGEMDELKRKINQMVYNLRDSIQRNTQAREAAELANKTKSEFLANMSHEIRTPMNGIIGMTQLTLDTDLTQYQREMLNIVNNLAMSLLTIIDDILDLSKIEAKRMVIEEIPYTLRGTVFNALKTLAVKANDKFLDLTYRVDSSVPDHVIGDSFRLRQIILNLVGNAIKFTEHGEVSLTIQKGNDVTCLPNEYMIEFVVSDTGIGIPTDKLGLIFDTFQQADGSMTRKFGGTGLGLSISKRLVNLMGGDVWVKSQYGKGSSFYFTCRVRLADVDISLIRKQLKPYKGHQVLFIDKGKTGHGPEVGQMLGQLGLVPIVLESEQNHTLTRVRGKECPYDVIVVDSIDTARRLRGIDDFKYLPIVLLAPTVHVSLKSCLDLGITSYMTMPCKLIDLGNGMVPALENRATPSLSDNTKSFEILLAEDNTVNQRLAVKILEKYNHVVTVVSNGAEALEAVKDNKYDVILMDVQMPVMGGFEATAKIREYERSLGTQRTPIIALTAHAMMGDREKCIEAQMDEYLSKPLQQNHLIQTILKCATLGGALLEQNRERELELARHAEHKGGLSTDPARASSVMRPPLHHRPVTTAESLSGGAESPSLMANDGEDPIQRARSSLSEPGCL

SEQ ID NO: 17 - protein, 1298 aa, Neurospora crassa
MTDGPTLAAIAALVKSLAVDPATTQTSGLRPSTHVRLPGPYTREKGDLERELSALVVRIEQLETAAIAASPPAMPDTPNAPTDALFSNGTLSPSSETPDARYPAPLPRNGFIDEALEGLREHVDDQSKLLDSQRQELAGVNAQLIEQKQLQEKALAIIEQERVATLERELWKHQKANEAFQKALREIGEIVTAVARGDLSKKVRMNSVEMDPEITTFKRTINTMMDQLQVFSSEVSRVAREVGTEGILGGQAQIEGVDGTWKELTDNVNVMAQNLTDQVREIASVTTAVAHGDLTKKIERPAKGEILQLQQTINTMVDQLRTFASEVTRVARDVGTEGILGGQADVEGVQGMWNELTVNVNAMANNLTTQVRDIIKVTTAVAKGDLTQKVQAECRGEIFELKKTINSMVDQLQQFAREVTKIAREVGTEGRLGGQATVHDVQGTWRDLTENVNGMAMNLTTQVREIAKVTTAVAKGDLTKKIGVEVQGEILDLKNTINTMVDRLGTFAFEVSKVAREVGTDGTLGGQAQVDNVEGKWKDLTENVNTMASNLTSQVRGISTVTQAIANGDMSRKIEVEAKGEILILKETINNMVDRLSIFCNEVQRVAKDVGVDGIMGGQADVAGLKGRWKEITTDVNTMANNLTAQVRAFGDITNAATDGDFTKLVEVEASGEMDELKKKINQMVYNLRDSIQRNTQAREAAELANKTKSEFLANMSHEIRTPMNGIIGMTQLTLDTDLTQYQREMLNIVNSLANSLLTIIDDILDLSKIEARRMVIEEIPYTLRGTVFNALKTLAVKETEKFLDLTYRVDHSVPDHVVGDSFRLRQIILNLVGNAIKFTEHGEVSLTIQKASSVQCSTEEYAIEFVVSDTGIGIPADKLDLIFDTFQQADGSMTRKFGGTGLGLSISKRLVNLMGGDVWVKSEYGKGSKFFFTCVVRLANDDISLIAKQLNPYKSHQVLFIDKGRTGHGPEIAKMLHGLGLVPIVVDSERNPALEKARAAGQAPYDVIIVDSIEDARRLRSVDDFKYLPIVLLAPVVHVSLKSCLDLGITSYMTTPCQLIDLGNGMVPALENRATPSLADNTKSFEILLAEDNTVNQRLAVKILEKYHHVVTVVGNGEEAVEAVKRKKFDVILMDVQMPIMGGFEATAKIREYERSLGSQRTPIIALTAHAMMGDREKCIQAQMDEYLSKPLQQNHLIQTILKCATLGGQLLEKNRERELTRAADAVTGGRRDNGMYSASQAAQHAALRPPLATRGLTAADSLVSGLESPSIVTADKEDPLSRARASLSEPNIHKAS